(19) United States
(12) Patent Application Publication
(10) Pub. No.: US 2025/0002882 A1
(43) Pub. Date: Jan. 2, 2025

(54) CPF1 PROTEIN AND ITS USE IN GENE EDITING

(71) Applicant: ZHEJIANG LAB, HANGZHOU (CN)

(72) Inventors: JIN TANG, HANGZHOU (CN); JUNCHAO SHI, HANGZHOU (CN); YUANYUAN FENG, HANGZHOU (CN); PEIXIANG MA, HANGZHOU (CN); XINGXU HUANG, HANGZHOU (CN)

(21) Appl. No.: 18/658,938

(22) Filed: May 8, 2024

(30) Foreign Application Priority Data
Mar. 10, 2023 (CN) 202310258665.1

(51) Int. Cl. C12N 9/22; C12N 15/11; C12N 15/63

(52) U.S. Cl. CPC C12N 9/22; C12N 15/111; C12N 15/63; C12N 2310/20; C12N 2800/22

(57) ABSTRACT

The present invention discloses a Cpf 1 protein and its use in gene editing, which relates to the field of biotechnology. The present invention provides a novel Cas12a nuclease named as the Cas12a-68 protease, which has a wider PAM recognition range than traditional nucleases and is capable of recognizing targets of various genes. When editing nucleic acid fragments, using the new nuclease of the present invention is capable of overcoming the shortcomings of the traditional Cas12a and increasing the PAM recognition range. These advantages make the present invention more suitable for editing a wider range of nucleic acid fragments and lay a foundation for gene editing applications.

This application claims priority of Chinese Application No. 202310258665.1, filed Mar. 10, 2023, which is hereby incorporated by reference.

### FIELD OF TECHNOLOGY

The present invention relates to the field of biotechnology, in particular to a Cpf1 protein and its use in gene editing.

### BACKGROUND TECHNOLOGY

Gene editing technology enables the modification of DNA sequence loci, such as with the first-generation gene editing tool, zinc finger nucleases (ZFNs), and the second-generation tool, transcription activator-like effector nucleases (TALENs), can both be used to modify targeted genomes, but these methods are difficult to design, not easy to manufacture, expensive, and not universal.

CRISPR-Cas systems originate from the RNA-guided adaptive immune systems of bacteria, initially utilized to combat nucleic acid invaders such as bacteria and archaea. CRISPR refers to clustered regularly interspaced short palindromic repeats, which are derived from phage DNA fragments that are capable of infecting prokaryotes. CRISPR are capable of detecting and destroying similar DNA in other phages that can cause similar infections, so they are critical for prokaryotes to resist phages. Therefore, the sequence is the immune system of many prokaryotes. Cas proteins refer to CRISPR-associated proteins, which are associated proteins in CRISPR systems. A CRISPR locus consists of an operon encoding a Cas protein and a repeat spacer, which can be guided by RNA corresponding to the spacer in the CRISPR sequence, to recognize and cleave specific DNA strands complementary to its sequence. The CRISPR/Cas9 system belongs to the class 2 CRISPR system, which consists of Cas9 nuclease, crRNA and tracrRNA, and is the most widely used gene editing tool currently; this system is simple and efficient, and only needs to synthesize a new segment of RNA to realize gene editing, and its properties of simple design and easy operation have become the greatest advantages, making this technology a convenient and adaptable tool for regulating and observing genomes. This allows the system to be applied in biological research and biotechnology in a wide range of fields, including different fields such as gene regulation, surface modification, base editing, gene imaging and so on, and has shown a wide range of applicability in microorganisms, plants, animals and even human bodies. The CRISPR-Cas tool has greatly accelerated the pace of scientific research, and the research and development of biotechnology based on Cas has also made rapid progress. Several clinical trials leveraging Cas9 are either ongoing or imminent, with their outcomes expected to inform future applications of somatic gene editing in vitro and in clinical settings.

Currently, the CRISPR/Cas system has developed from the initial CRISPR/Cas9 to various gene editing systems such as the present CRISPR/Cas12a, and CRISPR/Cas13a. Among them, CRISPR-Cas12a is a Class 2 RNA-guided endonuclease, which can be used to edit the CRISPR system of mammalian genomes. In September 2015, Zhang Feng's group (Zetsche B, Gootenberg J S, Abudayyeh 0, et al. Cpf1 is a single RNA-guided endonuclease of a class 2 CRISPR-Cas system. Cell, 2015, 163(3): 759-771.) added a new member to the CRISPR family-Cpf1, which belongs to type V of the class 2 CRISPR-Cas system. The CRISPR/Cpf1 system belongs to type V of class 2, which is small in size, has the abilities of both RNA endonuclease and DNA endonuclease, and can recognize PAM sequences rich in thymine (T), thus expanding the editable range of the CRISPR/Cas system. The system not only has good activity of targeted cleavage of DNA, but also shows editing characteristics that are significantly different from CRISPR-Cas9. The Cas12a nuclease enlarges the selection range of gene editing target sites, while the off-target effect is lower. Compared with the Cas9 nuclease, the Cas12a nuclease recognizes the DNA target sequences complementary to crRNA spacers without using tracrRNA. Moreover, the Cas12a protease has a smaller molecular weight, and is easier to enter cells, so the editing success rate is higher. Cleavage with a Cpf1 protein only needs the guidance of a single RNA, which can simplify the design and use of gene editing tools. Meanwhile, the system Cpf1 produces the effect of gene editing by recognizing T-rich PAM and leaving a cohesive end in its targeted DNA sequence. The TIC preference of a Cpf1 family protein to recognize PAM extends the range of targeted editing nucleic acids with CRISPR. However, gene editing with a traditional Cas12a protein is limited by the protospacer adjacent motif (PAM), and only a portion of nucleic acid targets can be recognized, for example, LbCas12a tends to recognize the PAM sequence containing TTTV; Acidaminococcus Cpf1 (AsCpf1) and Lachnospiraceae Cpf1 (LbCpf1) are widely used in the world, and because of their lack of flexibility in recognizing the protospacer adjacent motif of 5′-TTTN-3′, their application in the field of gene editing is limited. Therefore, it is necessary to find a Cas12a protein or improve the existing Cas12a protein (which refers to a crRNA-dependent endonuclease, also known as the Cpf1 protein, which is a type V enzyme in CRISPR system classification), and find a novel Cas12a protease which has a wider recognition range and is more convenient to use.

### SUMMARY OF THE INVENTION

The technical problem to be solved by the present invention is to overcome the defect that the range of PAM of dsDNA target sequences recognized by Cas12a is limited in the prior art. Provided in the present invention is a novel Cas12a nuclease named as the Cas12a-68 protease, which has a wider PAM recognition range than traditional nucleases and is capable of recognizing targets of various genes.

A novel Cas12a nuclease, i.e., a Cpf1 protein, wherein an amino acid sequence of the Cpf1 protein is shown as SEQ ID NO.1.

Also provided in the present invention is a polynucleotide encoding the Cpf1 protein.

Cpf1 (Cas12a) is capable of recognizing a DNA nucleic acid sequence under the guidance of RNA, but the PAM recognition range of a traditional Cas12a protein is limited, for example, LbCas12a PAM is TTTV, and the novel Cas12a-68 protease we found has a wider PAM recognition range and expands the recognition range.

Preferably, the polynucleotide is codon optimized for expression in a target cell.

Also provided in the present invention is a vector comprising the polynucleotide.

The Cpf1 protein is a codon-optimized Cpf1 protein. The nucleic acid sequences of Cpf1 proteins from different sources are prokaryotic codon optimized to obtain the sequences, constructed into the pET28a expression vector to induce the expression of soluble proteins at low temperature, and purified by affinity purification and molecular sieve purification to obtain the target protein.

Also provided in the present invention is a type V CRISPR/Cas 12a gene editing system, comprising the Cpf1 protein or a polynucleotide encoding the Cpf1 protein.

The type V CRISPR/Cas 12a gene editing system further comprises CRISPR RNA. A sequence of the CRISPR RNA is shown as at least one of SEQ ID NO.7-10.

Also provided in the present invention is the use of the Cpf1 protein, the polynucleotide, the vector, or the gene editing system in gene editing.

The use is binding to, targeted cleavage or non-targeted cleavage of DNA.

Also provided in the present invention is a gene editing method that utilizes the gene editing system to edit a target DNA.

Specifically, the method includes the following steps:
(1) designing CRISPR RNA for a target gene sequence to be edited;
(2) constructing a gene editing vector comprising an encoding gene sequence of the CRISPR RNA and the polynucleotide; and
(3) introducing the gene editing vector in step (2) into a receptor cell to be gene edited, and screening to obtain a gene edited transgenic cell.

In the present invention, once a target nucleic acid, crRNA and a Cas12a protein form a ternary complex, the cleavage activity of the Cas12a protease will be activated, and the target DNA will be cleaved by Cas12a. In the presence of suitable PAM, different target DNA fragments can be targeted by designing different crRNA sequences, and the principle is shown in FIG. 1.

In the present invention, the target DNA molecule is inserted into the pET-28a vector and linearized with an EcoR V endonuclease. During the identification experiment of gene editing, in the presence of PAM recognized by the Cas12a protease, the protease will cleave the target DNA molecule from the linearized vector by adding the corresponding crRNA. The reaction system is heated at 95° C. for 5-10 minutes to inactivate the Cas12a protease, and then the target DNA molecule cleaved is observed by agarose gel electrophoresis. Furthermore, we also find that the active sites of the novel Cas12a-68 protease are D897 and E982 by the following analysis: mutating either D897 or E982 of the Cas12a-68 expression vector to alanine, and then assessing whether the mutant can cleave the target dsDNA according to the above steps.

### Description of Terms

The term crRNA refers to CRISPR RNA, which is a short RNA that guides Cas12a (Cpf1) to bind to the target DNA sequence.

The term CRISPR refers to clustered regularly interspaced short palindromic repeats, and the sequence is the immune system of many prokaryotes.

The term Cas protein refers to CRISPR-associated proteins, which are associated proteins in CRISPR systems.

The term Cpf1 (also called Cas12a) refers to the crRNA-dependent endonuclease, which is a type V enzyme in CRISPR system classification.

The term PAM refers to the protospacer-adjacent motif, which is necessary for the cleavage with Cas12a. The PAM of FnCas12a is the TTN sequence, and the PAM of LbCas12a is the TTTN sequence.

Based on general knowledge of the art, the preferred conditions above can be arbitrarily combined to obtain various preferred examples of the present invention.

The reagents and raw materials used in the present invention are all commercially available.

The present invention has the following beneficial effects:

Provided in the present invention is a novel Cas12a nuclease named as the Cas12a-68 protease, which has a wider PAM recognition range than traditional nucleases and is capable of recognizing targets of various genes. When editing nucleic acid fragments, using the new nuclease of the present invention is capable of overcoming the shortcomings of the traditional Cas12a and increasing the PAM recognition range. These advantages make the present invention more suitable for editing a wider range of nucleic acid fragments and lay a foundation for gene editing applications.

### BRIEF DESCRIPTION OF THE DRAWINGS

FIG. 1 is a schematic diagram of gene editing with a programmable nuclease.

FIG. 2 is a detection diagram of the expression of a programmable nuclease in cells.

FIG. 3 is a detection diagram of the activity of a programmable nuclease to cleave double-stranded DNA.

FIG. 4 is a diagram showing the effect of different PAM fragments on gene editing with a programmable nuclease in cells.

FIG. 5 is a detection diagram of the effect of different nuclease mutations on nuclease cleavage of double strands.

### DESCRIPTION OF THE EMBODIMENTS

The present invention will be further elaborated in combination with specific examples. It should be understood that these examples are only used to illustrate the present invention and are not used to limit the scope of the present invention. Furthermore, it should be understood that after reading the teaching of the present invention, those skilled in the art can make various alterations or modifications to the present invention, and these equivalents also fall within the scope defined by the appended claims of the present application.

The experimental methods for which specific conditions are not specified in the following examples should be selected according to the conventional methods and conditions, or according to the product specifications.

In the present invention, conventional reagents such as Tris-Base (i.e., Tris base, also known as tris(hydroxymethyl)aminomethane), bovine serum albumin (BSA), NaCl, Tris-HCl (i.e., Tris hydrochloride, alias: tris-hydroxymethylaminomethane hydrochloride) and glycerol were purchased from China National Pharmaceutical Group Corporation; the nucleic acid fragments and RNA for detection were synthesized by Beijing Qingke Biotechnology Co., Ltd.; the transfection reagents used to transfect mammalian cells were purchased from Shanghai Li Ji Biotechnology Co., Ltd.; the FALG tag antibody used in the western-blot experiment was purchased from Cell Signaling Technology, Inc (CST); in the experiment, the second-generation sequencing experiment was completed by Annoroad Gene Technology (Beijing) Co., Ltd.; and the cell sorter used in the experiment was an ultra-high speed flow cytometry sorting system (BD FACSAria III).

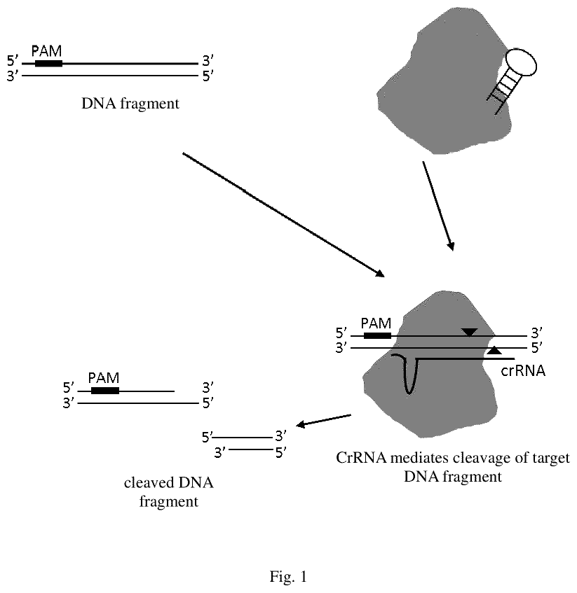

The gene editing principle of the programmable nuclease provided by the present invention is shown in FIG. 1. Specifically, when a Cas protease recognizes the existence of a PAM sequence, the complex formed by crRNA and the Cas12a protein targets the DNA sequence complementary to the bases of crRNA to open the DNA double strands, and crRNA binds to one of the single strands, i.e., the target strand (TS), at which time the cleavage activity of the Cas12a protease will be activated, and the target strand (TS) and the non-target strand (NTS) will be cleaved by the Cas12a protease. In the presence of suitable PAM, different target DNA fragments can be targeted by designing different crRNA sequences.

The present invention will be further elaborated in combination with specific examples.

### Example 1: Cloning and Intracellular Expression of Cas12a-68 Protease

In order to verify whether the novel Cas12a-68 protease has gene editing activity in mammalian cells, it was first verified whether Cas12a-68 was expressed in 293 cells (the human embryonic kidney cell 293, a cell line derived from human embryonic kidney cells).

Wherein, 293 cells have the following characteristics, for example: 1) they have high transfection efficiency; 2) are capable of being cultured in suspension for large-scale expression; 3) the structure of the protein expressed is closest to its conformation in human body; and 4) HEK293 cells can grow rapidly in the culture system and be cultured in high density. 293T cell line is a derivative cell line with high transfection efficiency formed by transfecting a SV40T-antigen gene into 293 cells. This enables the plasmid containing SV40ori to be significantly amplified in this cell line, thus promoting the amplification of expression vectors and the expression of proteins.

Expression of Cas12a-68 protease in HEK293T cells.

The gene fragment (the gene sequence is shown as SEQ ID NO.2) was cloned, and the amino acid sequence of Cas12a-68 used in the experiment was shown as SEQ ID NO.1. The gene fragment was amplified by PCR, and the pST1374 vector (Addgene, #13426) was digested by Not I, and then the gene fragment and vector were recombined under the action of recombinase (Vazyme, C112-01). The recombinant plasmid vector was identified by sequencing, and then the plasmid was extracted. The plasmid was transfected into 293 cells by the EZ Trans cell transfection reagent (Li Ji Biotechnology Co., Ltd., AC04L091/AC04L092) and cultured at 37° C. for 48-72 hours. The cells were collected, lysed, and treated by SDS-PAGE protein electrophoresis, and then detected by Western-blot.

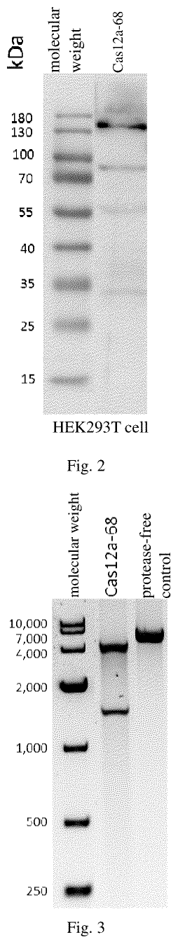

The experimental results of the Western-blot detection are shown in FIG. 2. The results show that the Cas12a-68 protein can be expressed in mammalian cells.

### Example 2: Cas12a-68 Protease has In Vitro Gene Cleavage Activity

### 1. Preparation of Nucleic Acid

CrRNAs were synthesized by Beijing Qingke Biotechnology Co., Ltd., and the sequences of crRNAs synthesized are shown in Table 1.

TABLE 1

| crRNA | sequence (5′-3′) |
|---|---|
| Mers-cr2+ | UaaUUUcUacUaagUgUagaUGACAUAUGGAAAACGA |
| | ACUAUGU (SEQ ID. NO. 6) |

### 2. Detection of Gene Cleavage Activity In Vitro Based on CRISPR/Cas12a

In the experiment, crRNA consisted of a 21 nt region interacting with Cas12a and a 23 nt programmable guiding region for target DNA recognition. The target DNA was a gene fragment of the MERS virus (the sequence used in the experiment was shown as SEQ ID NO.3). The target DNA fragment was amplified by PCR with forward and backward primers with Nco I and EcoR I enzymes respectively. Then the pET28a vector was double digested by Nco I and EcoR I. The digested vector and the PCR amplification product were reconnected under the action of the T4 ligase, and the ligation product was used to transform DH5a competent cells. The monoclonal strain was selected and cultured by shaking, before collecting the bacteria to extract the plasmid vector. The extracted plasmid was linearized with the EcoR V endonuclease for the detection reaction.

The detection system used was 400 ng of Cas12a-68, 100 ng of target DNA fragments (the gene fragment of MERS virus, the sequence of which was shown as SEQ ID NO.3), and an amount of 5 pmol of crRNA, with the final volume of the reaction of 10 μL. The reaction was incubated at 37° C. for one hour, and heated at 95° C. for 5-10 minutes to inactivate the Cas12a protease. Then, the target DNA molecule cleaved was observed by agarose gel electrophoresis. After a linearized plasmid is cleaved by Cas, two DNA fragments with lengths of about 4100 bp and 1400 bp will be formed.

In a gene editing system, once crRNA mediates a Cas protease to target a target DNA molecule, the cleavage activity of the protease will be activated, and then the target DNA fragment will be cleaved. The specific experimental results are shown in FIG. 3. From FIG. 3, it can be seen that Cas12a-68 is capable of cleaving double-stranded DNA molecules during the in vitro cleavage activity experiment, indicating that the Cas12a-68 protease has gene editing activity in vitro.

### Example 3: Efficiency of Cas12a-68 Protease in Cleaving Fragments Containing Different PAM

According to the results of Example 1, the Cas12a-68 protease can be expressed in mammalian cells. This applicant identified whether the protease also had gene editing ability in cells, as well as identified whether different PAM affected the cleavage with the Cas12a-68 protease in cells.

In the experiment, there were four crRNA plasmids, i.e., the libraries where N in NNNN in PAM sequences was A, T, G and C, respectively. The crRNA sequences expressed by the four plasmids are shown in Table 2. In this experiment, crRNA consisted of a 21 nt region interacting with Cas12a and a 20 nt programmable guiding region for target DNA recognition.

TABLE 2

| crRNA | sequence (5′-3′) |
|---|---|
| NNNA-crRNA | UaaUUUcUacUaagUgUagaUccggUUccggcaccgggcUU (SEQ |
| | ID. NO. 7) |
| NNNT-crRNA | UaaUUUcUacUaagUgUagaUggacatCgaagaagacatcC (SEQ ID. |
| | NO. 8) |
| NNNC-crRNA | UaaUUUcUacUaagUgUagaUccagagcaUcccgUggaacG (SEQ ID. |
| | NO. 9) |
| NNNG-crRNA | UaaUUUcUacUaagUgUagaUgaUgUcUUcUUcGaUgUccA(SEQ |
| | ID. NO. 10) |

The cell lines used in the experiment contained plasmid libraries with different PAM sequences. As shown in Table 3, PAM has TNNN and CNNN, while the complementary chains have different PAM sequences of NNNA and NNNG, where N represents any base, i.e., A, T, G and C.

TABLE 3

| target | |
|---|---|
| DNA | sequence (5′-3′, where N represents any base) |
| NNNN | gccttcctggagacctccgcgccccgcaacctccccttctacgagcggctcggcttcaccgtcaccgc |
| (the cell | cgacgtcgaggtgcccgaaggaccgcgcacctggtgcatgacccgcaagcccggtgccggaAcc |
| plasmid | ggTNNNTggacatCgaagaagacatcCNNNCccagagcatcccgtggaacGTAACT |
| vector | ggatccggcgcaacaaacttctctctgctgaaacaagccggagatgtcgaagagaatcctggacTgg |
| cleavage | tgagcaagggcgaggagctgttcaccggggtggt (SEQ ID. NO. 11) |
| sequence) | |

The cell lines used in the experiment contained plasmid libraries with different PAM sequences, and the cleavage region was located in front of the green fluorescent protein (GFP) gene. Because of frame shift mutation (which refers to the change of the reading frame of a DNA molecule due to the deletion or insertion of a base at a certain site, which leads to a series of code changes downstream, so that the gene originally encoding a certain peptide chain becomes a sequence encoding a completely different peptide chain), GFP could not be expressed, and there was no fluorescence under laser. However, in cells, when a Cas12a-68 protease binds to crRNA and targets a gene region with a suitable PAM sequence, the Cas12a-68 protease cleavage activity will be activated to cleave the gene fragment in the target region. Theoretically, because of the randomness of cleavage, the cleavage reaction has a ⅓ probability of making the base sequence in front of GFP in-frame, which allows GFP to be expressed and emit green fluorescence.

In the experiment, the four crRNA plasmid libraries were co-transfected with the Cas12a-68 pST1374 plasmid constructed in Example 1, respectively, into cells containing plasmid libraries with different PAM sequences. Puro antibiotic (puromycin, a protein synthesis inhibitor) was added for stress screening, and the cells were collected when the culture dish was full (about 6 days). Then, the cells were sorted, and the cells that could express the green fluorescent protein were collected. After that, the cells were broken, the genome was extracted, and four pairs of different primers were used to amplify the cleavage region fragment. The primer sequences are shown in Table 4. The PCR products were sent to Annoroad Gene Technology (Beijing) Co., Ltd. for second-generation sequencing, and then the sequencing results were analyzed.

TABLE 4

| primer | sequence (5′-3′) |
|---|---|
| HG-crRNAseq-F | agtcaaaacctccccttctacgagcg |
| | (SEQ ID. NO. 12) |
| NNNA-crRNAseq-R1 | agctcctcgcccttgctcac (SEQ |
| | ID. NO. 13) |
| NNNT-crRNAseq-R2 | acttgaagctcctcgcccttgctcac |
| | (SEQ ID. NO. 14) |
| NNNG-crRNAseq-R3 | gatcagagctcctcgcccttgctcac |
| | (SEQ ID. NO. 15) |
| NNNC-crRNAseq-R4 | tagcttagctcctcgcccttgctcac |
| | (SEQ ID. NO. 16) |

According to the data of the second-generation sequencing, we analyzed the number of cleavage reactions under each PAM sequence and made a heat map. The darker the color, the higher the cleavage efficiency.

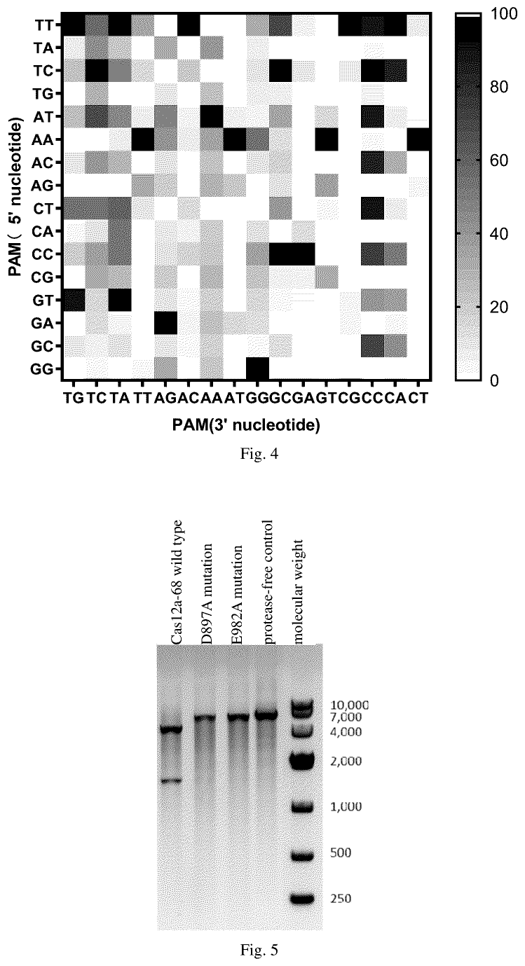

The results are shown in FIG. 4. According to the experimental results, besides the PAM sequence with LbCas12a TTTV, Cas12a-68 has a wider PAM recognition range, such as NYCC, TTCV, GTTR, AANT, CCGM, etc. (R represents A+G; Y represents C+T; M represents A+C; N represents A+C+G+T; and V represents A+C+G). The experimental results are shown in FIG. 4, which shows that the Cas12a-68 protease can exert its cleavage activity in mammalian cells, and has a wider PAM recognition range.

### Example 4: Effect of Different Mutations of Cas12a-68 Protease on Cleavage Activity

By analyzing the sequence conservation, we found the potential protease cleavage active sites D897 and E982. In order to verify whether the sites affect the cleavage activity of the Cas12a-68 protease, we constructed site mutation plasmids of D897A and E982A respectively on the basis of the Cas12-68 pET28a plasmid vector. The PCR primers for site mutation were synthesized, and the primer sequences are shown in Table 5. Then the plasmid vector was used as a template for PCR reaction, and the reaction products were used to transform DH5a competent cells. The monoclonal strains were selected, and then sent to be sequenced to identify whether the sites were mutated correctly.

TABLE 5

| primer | sequence (5′-3′) |
|---|---|
| 68_D897A-F | TGTGAACATTATCGGCATCGCTCGTGGCGAGCGTAA |
| | TCT (SEQ ID. NO. 17) |
| 68_D897A-R | CGATGCCGATAATGTTCACATCAGAGCAGTCCTTCA |
| | CG (SEQ ID. NO. 18) |
| 68_E982A-F | TAAGGCAATCGTGGTCCTTGCAAATTTAAACGTGGG |
| | GTT (SEQ ID. NO. 19) |
| 68_E982A-R | CAAGGACCACGATTGCCTTATACTTTACAATCATCT |
| | TG (SEQ ID. NO. 20) |

Then two plasmid vectors with single site mutation were used to transform BL21 (DE3) competent cells to extract the protein containing site mutation. The Cas12a-68 site mutation protein was expressed in E. coli. The bacteria in the medium were collected, lysed by ultrasound, and purified by nickel column affinity chromatography with the target protein eluted with different concentrations of imidazole. The eluent was collected, and desalted by a desalting column, which could be used to detect the reaction.

According to the detection method of in vitro cleavage activity of proteases in Example 2, that is, in the experiment, crRNA consisted of a 21 nt region interacting with Cas12a and a 23 nt programmable guiding region for target DNA recognition. The target DNA was a gene fragment of the MERS virus (the gene sequence was shown as SEQ ID NO.3). The target DNA fragment was amplified by PCR with forward and backward primers with Nco I and EcoR I enzymes respectively. Then the pET28a vector was double digested by Nco I and EcoR I. The digested vector and the PCR amplification product were reconnected under the action of the T4 ligase, and the ligation product was used to transform DH5a competent cells. The monoclonal strains were selected, and the fragment was identified to be correctly inserted into the vector by sequencing. Then the plasmid vector was extracted, and linearized with the EcoR V endonuclease for the detection reaction.

The detection system used was 400 ng of Cas12a-68-D897A or Cas12a-68-E982A (Cas12a-68-D897A was a sequence in which the 897^{th} amino acid of the wild-type Cas12a-68 amino acid sequence was mutated from aspartic acid into alanine, and the amino acid sequence was shown as SEQ ID NO.4; and Cas12a-68-E982A was a sequence in which the 982^{nd} amino acid of the wild-type Cas12a-68 amino acid sequence was mutated from glutamic acid into alanine, and the amino acid sequence was shown as SEQ ID NO.5), 100 ng of target DNA fragments (the gene fragment of MERS virus, the sequence of which was shown as SEQ ID NO.3), and an amount of 5 pmol of crRNA, with the final volume of the reaction of 10 μL. The reaction was incubated at 37° C. for one hour, and heated at 95° C. for 5-10 minutes to inactivate the Cas12a protease. Then, agarose gel electrophoresis was performed to observe the cleaved target DNA molecules. Whether the site mutation protease had cleavage activity in vitro was verified.

Conclusion: The experimental results are shown in FIG. 5, which shows that the two single point mutations of D897A and E982A lose their cleavage activity in vitro, indicating that D897 and E982 are critical for the cleavage activity of the Cas12a-68 protease.

The above is only the preferred embodiment of the present invention, but the protection scope of the present invention is not limited to this. Anyone skilled in the art can make equivalent replacements or changes according to the technical scheme of the present invention and the inventive concept thereof within the technical scope disclosed by the present invention, which should be all covered by the protection scope of the present invention.

The replacement Sequence Listing is in XML format and is hereby incorporated by reference. The name of the XML file is SEQ_LISTINGS_Replacement_File4 with the date of creation of Sep. 20, 2024 and size of 1,048,576 bytes. The replacement “Sequence Listing XML” does not include new matter

1. A Cpf1 protein, wherein the amino acid sequence of the Cpf1 protein is shown as SEQ ID NO.1.

2. A polynucleotide encoding the Cpf1 protein of claim 1.

3. The polynucleotide of claim 2, wherein the polynucleotide is codon optimized for expression in a target cell.

4. A vector comprising the polynucleotide of claim 2.

5. A type V CRISPR/Cas 12a gene editing system, comprising the Cpf1 protein of claim 1 or a polynucleotide encoding the Cpf1 protein of claim 1.

6. The type V CRISPR/Cas 12a gene editing system of claim 5, further comprising CRISPR RNA.

7. The type V CRISPR/Cas 12a gene editing system of claim 6, wherein a sequence of the CRISPR RNA is shown as at least one of SEQ ID NO.7-10.

8. A method of gene editing comprising the step of utilizing at least one of the following:
(a) the Cpf1 protein of claim 1;
(b) a, polynucleotide encoding the Cpf1 protein;
(c) a vector comprising the polynucleotide; or
(d) a type V CRISPR/Cas 12a gene editing system comprising the Cpf1 protein or the polynucleotide encoding the Cpf1 protein.

9. The method of gene editing of claim 8, wherein the method comprising the step of binding to, targeted cleavage or non-targeted cleavage of DNA.

10. A gene editing method, comprising the following steps:
(1) designing CRISPR RNA for a target gene sequence to be edited;
(2) constructing a gene editing vector comprising an encoding gene sequence of the CRISPR RNA and the polynucleotide of claim 2; and
(3) introducing the gene editing vector in step (2) into a receptor cell to be gene edited, and screening to obtain a gene edited transgenic cell.

11. A gene editing method, comprising the following steps:
(1) designing CRISPR RNA for a target gene sequence to be edited;
(2) constructing a gene editing vector comprising an encoding gene sequence of the CRISPR RNA and the polynucleotide of claim 3; and
(3) introducing the gene editing vector in step (2) into a receptor cell to be gene edited, and screening to obtain a gene edited transgenic cell.

12. The method of gene editing of claim 8, wherein the polynucleotide is codon optimized for expression in a target cell.

13. The method of gene editing of claim 8, wherein the type V CRISPR/Cas 12a gene editing system further comprises CRISPR RNA.

14. The method of gene editing of claim 13, wherein a sequence of the CRISPR RNA is shown as at least one of SEQ ID NO.7-10.

15. A vector comprising the polynucleotide of claim 3.


SEQUENCE LISTING (20 sequences)

SEQ ID NO: 1 - protein, 1293 aa, synthetic construct
MRTMVTFENFTKQYQVSKTLRFELIPQGKTLENMKRDGIISVDRQRNDDYQKAKGILDKLYKYILDSTMETAVIDWEELAIAIEEFRKSKDKKTYEKVQSKVRTALLEHVKKQKVGTEDLFKGMFSSKIITGEVLAAFPEIRLSDEENLILEKFKDFTTYFTGFFENRKNVFTDEALSTSFTYRLVNDNFIKFFDNCTVMKNVVNISPDMAKSLETCVSDLGIFPGVSLEEVFSVSFYNRLLTQTGIDQFNQLLGGISGKEGEYKKQGLNEIINLAMQQSPEVKEVLKNKAHRFTPLFKQILSDRSTMSFIPDAFADDDEVLSAVDAYRKYLLEKNIGDRAFQLISDIEEYSPELMRIGGKYVSVLSQLLFNSWSEIRDGVKAYKESLITGKKTKKELENIDKGIKYGVTLQEIKEALPKKDIYEEVKKYAMSVVKDYHAGLAEPLPEKIETDDERASIKHIMDSMLGLYRFLEYFSHDSIEDTDPVFGECLDTILDDMNETVPLYNKVRNFSTRKVYSTEKFKLNFNNSSLANGWDKNKEQANGAVLLKKAGEYFLGIFNSKNKPKLVSDGGGGTGYEKMIYKQFPDFKKMLPKCTISRKETKAHFQKSDEDFTLLTDKFEKSLVITKKIYDLGTQTVNGKKKFQVDYPRLTGDMEGYRAALKEWIDFGKKFIQAYASTAIYDTSLFRNSSDYPDLPSFYKDVDNICYKLTFECIPDAVINDCIDDGSLYLFKLHNKDFSAGSIGKPNLHTLYWKAIFEEENLSDVVVKLNGQAELFYRPKSLTRPVVHEAGEVIINKTTSTGLPVPDDVYVELSKFVRNGKKGNLTDKAKNWLDKVTVRKTPHAIIKDRRFTVDKFFFHVPITLNYKADSSPYRFNDFVRQYVKDCSDVNIIGIDRGERNLIYAVVIDGKGNIIEQRSFNTVGTYNYQEKLEQKEKERQTARQDWATVTKIKDLKKGYLSAVVHELSKMIVKYKAIVVLENLNVGFKRMRGGIAERSVYQQFEKALIDKLNYLVFKDEEQSGYGGVLNAYQLTDKFESFSKMGQQTGFLFYVPAAYTSKIDPLTGFINPFSWKHVKNREDRRNFLNLFSKLYYDVNTHDFVLAYHHSNKDSKYTIKGNWEIADWDILIQENKEVFGKTGTPYCVGKRIVYMDDSTTGHNRMCAYYPHTELKKLLSEYGIEYTSGQDLLKIIQEFDDDKLVKGLFYIIKAALQMRNSNSETGEDYISSPIEGRPGICFDSRAEADTLPHNADANGAFHIAMKGLLLTERIRNDDKLAISNEEWLNYIQEMRG

SEQ ID NO: 2 - DNA, 3879 bp, synthetic construct
  source 1..3879: mol_type = other DNA
atgcgtactatggtaacgttcgagaacttcaccaaacaatatcaggtctctaagacgttgcgtttcgaacttatccctcaaggcaaaacattagagaatatgaaacgtgacggtattattagtgtggaccgccaacgcaacgacgattatcagaaggctaaaggcatcttggacaagttatacaagtacattttagattcaacgatggagacagcggtgatcgattgggaggaactggccattgcaatcgaggagttccgtaaatcaaaggataaaaagacatacgaaaaggttcaatcaaaggtccgcactgcccttttggagcatgtcaagaaacagaaagtgggcacagaggacttgtttaaagggatgttcagttcaaaaattattacaggagaagtcttggcagcatttcctgagattcgtttatctgacgaggagaatcttattttagagaagtttaaagacttcactacatactttactggattcttcgaaaaccgtaaaaatgttttcactgacgaagccttgagcactagcttcacttatcgtcttgtgaatgacaatttcatcaaatttttcgataattgcactgtgatgaaaaacgttgtgaacatctcgcccgacatggcgaagtcccttgagacatgcgtcagcgacttaggtattttcccgggtgtttccttagaggaagtgttttcggtcagtttttataatcgccttctgacacaaacaggcattgatcaatttaaccaattattaggcggcatttctggcaaggaaggcgaatataagaagcaaggactgaacgagatcatcaatttggcgatgcagcaatcccctgaagttaaggaggttttgaagaacaaggcccaccgctttacaccccttttcaaacaaattttatcggatcgttcgacaatgagtttcatcccggacgcgtttgctgacgatgatgaagttctttcagccgtggacgcatatcgtaagtatcttcttgaaaaaaatatcggtgaccgcgcctttcagttaatttcggacattgaggagtactcacctgagttaatgcgcatcgggggcaagtatgttagtgttttgagccaattgttgtttaactcatggtctgagatccgtgatggggtgaaggcatataaggaatcactgatcacaggaaaaaaaacaaagaaagagttggaaaacattgacaagggtatcaagtacggcgtgactcttcaggaaatcaaggaagctttaccaaaaaaagatatttacgaggaggtgaagaaatacgcgatgtccgtagtaaaagactatcatgccggcttagctgaaccgttgccagaaaaaatcgaaacggacgacgaacgtgcatccatcaaacatattatggattcaatgctgggactgtaccgcttcttagaatatttctcgcatgacagcattgaggacactgacccagtttttggagaatgcctggatacaattcttgatgacatgaacgagaccgttcctttgtataacaaggttcgcaatttctcgactcgtaaggtatattctacagaaaagttcaaattaaactttaacaactcaagtttggcaaacggatgggataaaaataaagaacaagctaatggtgctgtccttctgaaaaaggcgggggagtatttcttaggcatcttcaattctaagaacaagcccaagctggtatccgatggaggtggaggtacaggctatgagaaaatgatctataagcagtttcccgattttaagaagatgttacctaaatgtacaatcagccgcaaagagacaaaagcgcatttccagaaatcggacgaagacttcacgttattaacagacaaatttgagaagagcctggtaattaccaaaaagatctatgatttaggcactcaaacggtcaacggaaagaagaagtttcaagtcgactacccgcgccttaccggggatatggaggggtaccgcgctgcactgaaagaatggatcgattttggaaagaaatttattcaagcctacgcgtccactgcgatctatgatacttccctgtttcgtaattcctccgattatcctgacttgccatcattctacaaagatgttgataacatctgttataaattaactttcgaatgtattccagacgcagtcattaatgattgtatcgacgacgggagcttgtacttattcaaattgcacaataaggacttctcggccgggtccattggcaagccgaacctgcatactctttactggaaagcgatctttgaggaagagaatctttcggatgtggtagtcaaattgaatggtcaggccgagttgttctatcgtcccaaatcactgacgcgcccagtagttcatgaggctggtgaggtgattatcaacaagacgacaagcactggtttacctgttccagatgacgtctacgtcgagctttccaaatttgttcgcaacggtaaaaaaggtaatttaacggacaaagctaagaattggcttgacaaggtgactgtgcgtaagacgccccacgcgattatcaaagatcgtcgcttcactgtagacaagttcttcttccacgtgccaattactcttaattacaaggcggattcatccccttatcgctttaatgattttgtgcgtcagtacgtgaaggactgctctgatgtgaacattatcggcatcgatcgtggcgagcgtaatcttatctatgcagtcgttatcgacggaaagggtaatatcattgagcagcgcagctttaacaccgtagggacatataactatcaggaaaaattggaacaaaaggagaaagaacgtcagactgcacgccaagattgggccaccgtaaccaaaattaaggacctgaaaaaagggtacctgtcagcggtggttcatgaacttagcaagatgattgtaaagtataaggcaatcgtggtccttgaaaatttaaacgtggggttcaaacgtatgcgtggtggaattgcagaacgttctgtttaccagcagtttgagaaggcgttgatcgataaattgaattatttggtgtttaaggacgaagaacaatcaggttacggtggcgtgttaaacgcgtatcagcttacagacaagttcgagagtttcagcaagatgggccaacagaccgggtttttattctacgttcccgccgcatacacgtcaaagatcgatccactgaccggcttcattaaccctttcagctggaaacatgtaaaaaaccgcgaagaccgccgcaactttttgaacctgttcagcaaactgtattatgatgtcaacacacacgattttgtacttgcgtaccaccattccaacaaagattcaaaatatacaatcaaaggaaattgggaaatcgctgactgggatatcctgattcaagagaacaaagaggtgttcggtaagacggggacgccgtactgcgtcggaaagcgcattgtttatatggatgattcgacgacaggccacaaccgtatgtgtgcttactatccgcacaccgagttaaaaaaacttctgagcgaatacggtattgagtacacatctggccaagatcttttaaagattattcaagaatttgatgacgacaaattggtcaaaggcttgttttatatcattaaagcggcgctgcaaatgcgtaacagcaatagtgagacaggcgaagattacatttcatcccctatcgagggccgcccaggaatttgttttgactcgcgcgcagaggcggacacattacctcacaacgccgacgccaatggagccttccacatcgcgatgaaggggttgttgcttacagaacgtattcgcaatgatgataaattagcaatttctaatgaggagtggctgaactacatccaagagatgcgtggg

SEQ ID NO: 3 - DNA, 299 bp, unidentified
  source 1..299: mol_type = genomic DNA; note = coronaviridae(middle east respiratory syndrome-related coronavirus)
ccatgggctaccatgctgttttcgtgcctgcaacgcgcgattcagttcctcttcacataatcgccccgagctcgcttatcgtttaagcagctctgcgctactatgggtcccgtgtagaggctaatccattagtctctctttggacatatggaaaacgaactatgttaccctttgtccaagaacgaatagggttgttcatagtaaactttttcatttttaccgtagtatgtgctataacactcttggtgtgtatggctttccttacggctactagattatgtgtgtttttttgagaattc

SEQ ID NO: 4 - protein, 1293 aa, synthetic construct
MRTMVTFENFTKQYQVSKTLRFELIPQGKTLENMKRDGIISVDRQRNDDYQKAKGILDKLYKYILDSTMETAVIDWEELAIAIEEFRKSKDKKTYEKVQSKVRTALLEHVKKQKVGTEDLFKGMFSSKIITGEVLAAFPEIRLSDEENLILEKFKDFTTYFTGFFENRKNVFTDEALSTSFTYRLVNDNFIKFFDNCTVMKNVVNISPDMAKSLETCVSDLGIFPGVSLEEVFSVSFYNRLLTQTGIDQFNQLLGGISGKEGEYKKQGLNEIINLAMQQSPEVKEVLKNKAHRFTPLFKQILSDRSTMSFIPDAFADDDEVLSAVDAYRKYLLEKNIGDRAFQLISDIEEYSPELMRIGGKYVSVLSQLLFNSWSEIRDGVKAYKESLITGKKTKKELENIDKGIKYGVTLQEIKEALPKKDIYEEVKKYAMSVVKDYHAGLAEPLPEKIETDDERASIKHIMDSMLGLYRFLEYFSHDSIEDTDPVFGECLDTILDDMNETVPLYNKVRNFSTRKVYSTEKFKLNFNNSSLANGWDKNKEQANGAVLLKKAGEYFLGIFNSKNKPKLVSDGGGGTGYEKMIYKQFPDFKKMLPKCTISRKETKAHFQKSDEDFTLLTDKFEKSLVITKKIYDLGTQTVNGKKKFQVDYPRLTGDMEGYRAALKEWIDFGKKFIQAYASTAIYDTSLFRNSSDYPDLPSFYKDVDNICYKLTFECIPDAVINDCIDDGSLYLFKLHNKDFSAGSIGKPNLHTLYWKAIFEEENLSDVVVKLNGQAELFYRPKSLTRPVVHEAGEVIINKTTSTGLPVPDDVYVELSKFVRNGKKGNLTDKAKNWLDKVTVRKTPHAIIKDRRFTVDKFFFHVPITLNYKADSSPYRFNDFVRQYVKDCSDVNIIGIARGERNLIYAVVIDGKGNIIEQRSFNTVGTYNYQEKLEQKEKERQTARQDWATVTKIKDLKKGYLSAVVHELSKMIVKYKAIVVLENLNVGFKRMRGGIAERSVYQQFEKALIDKLNYLVFKDEEQSGYGGVLNAYQLTDKFESFSKMGQQTGFLFYVPAAYTSKIDPLTGFINPFSWKHVKNREDRRNFLNLFSKLYYDVNTHDFVLAYHHSNKDSKYTIKGNWEIADWDILIQENKEVFGKTGTPYCVGKRIVYMDDSTTGHNRMCAYYPHTELKKLLSEYGIEYTSGQDLLKIIQEFDDDKLVKGLFYIIKAALQMRNSNSETGEDYISSPIEGRPGICFDSRAEADTLPHNADANGAFHIAMKGLLLTERIRNDDKLAISNEEWLNYIQEMRG

SEQ ID NO: 5 - protein, 1293 aa, synthetic construct
MRTMVTFENFTKQYQVSKTLRFELIPQGKTLENMKRDGIISVDRQRNDDYQKAKGILDKLYKYILDSTMETAVIDWEELAIAIEEFRKSKDKKTYEKVQSKVRTALLEHVKKQKVGTEDLFKGMFSSKIITGEVLAAFPEIRLSDEENLILEKFKDFTTYFTGFFENRKNVFTDEALSTSFTYRLVNDNFIKFFDNCTVMKNVVNISPDMAKSLETCVSDLGIFPGVSLEEVFSVSFYNRLLTQTGIDQFNQLLGGISGKEGEYKKQGLNEIINLAMQQSPEVKEVLKNKAHRFTPLFKQILSDRSTMSFIPDAFADDDEVLSAVDAYRKYLLEKNIGDRAFQLISDIEEYSPELMRIGGKYVSVLSQLLFNSWSEIRDGVKAYKESLITGKKTKKELENIDKGIKYGVTLQEIKEALPKKDIYEEVKKYAMSVVKDYHAGLAEPLPEKIETDDERASIKHIMDSMLGLYRFLEYFSHDSIEDTDPVFGECLDTILDDMNETVPLYNKVRNFSTRKVYSTEKFKLNFNNSSLANGWDKNKEQANGAVLLKKAGEYFLGIFNSKNKPKLVSDGGGGTGYEKMIYKQFPDFKKMLPKCTISRKETKAHFQKSDEDFTLLTDKFEKSLVITKKIYDLGTQTVNGKKKFQVDYPRLTGDMEGYRAALKEWIDFGKKFIQAYASTAIYDTSLFRNSSDYPDLPSFYKDVDNICYKLTFECIPDAVINDCIDDGSLYLFKLHNKDFSAGSIGKPNLHTLYWKAIFEEENLSDVVVKLNGQAELFYRPKSLTRPVVHEAGEVIINKTTSTGLPVPDDVYVELSKFVRNGKKGNLTDKAKNWLDKVTVRKTPHAIIKDRRFTVDKFFFHVPITLNYKADSSPYRFNDFVRQYVKDCSDVNIIGIDRGERNLIYAVVIDGKGNIIEQRSFNTVGTYNYQEKLEQKEKERQTARQDWATVTKIKDLKKGYLSAVVHELSKMIVKYKAIVVLANLNVGFKRMRGGIAERSVYQQFEKALIDKLNYLVFKDEEQSGYGGVLNAYQLTDKFESFSKMGQQTGFLFYVPAAYTSKIDPLTGFINPFSWKHVKNREDRRNFLNLFSKLYYDVNTHDFVLAYHHSNKDSKYTIKGNWEIADWDILIQENKEVFGKTGTPYCVGKRIVYMDDSTTGHNRMCAYYPHTELKKLLSEYGIEYTSGQDLLKIIQEFDDDKLVKGLFYIIKAALQMRNSNSETGEDYISSPIEGRPGICFDSRAEADTLPHNADANGAFHIAMKGLLLTERIRNDDKLAISNEEWLNYIQEMRG

SEQ ID NO: 6 - RNA, 44 nt, synthetic construct
  source 1..44: mol_type = other RNA
taatttctactaagtgtagatgacatatggaaaacgaactatgt

SEQ ID NO: 7 - RNA, 41 nt, synthetic construct
  source 1..41: mol_type = other RNA
taatttctactaagtgtagatccggttccggcaccgggctt

SEQ ID NO: 8 - RNA, 41 nt, synthetic construct
  source 1..41: mol_type = other RNA
taatttctactaagtgtagatggacatcgaagaagacatcc

SEQ ID NO: 9 - RNA, 41 nt, synthetic construct
  source 1..41: mol_type = other RNA
taatttctactaagtgtagatccagagcatcccgtggaacg

SEQ ID NO: 10 - RNA, 41 nt, synthetic construct
  source 1..41: mol_type = other RNA
taatttctactaagtgtagatgatgtcttcttcgatgtcca

SEQ ID NO: 11 - DNA, 289 bp, synthetic construct
  source 1..289: mol_type = other DNA
gccttcctggagacctccgcgccccgcaacctccccttctacgagcggctcggcttcaccgtcaccgccgacgtcgaggtgcccgaaggaccgcgcacctggtgcatgacccgcaagcccggtgccggaaccggtnnntggacatcgaagaagacatccnnncccagagcatcccgtggaacgtaactggatccggcgcaacaaacttctctctgctgaaacaagccggagatgtcgaagagaatcctggactggtgagcaagggcgaggagctgttcaccggggtggt

SEQ ID NO: 12 - DNA, 26 bp, synthetic construct
  source 1..26: mol_type = other DNA
agtcaaaacctccccttctacgagcg

SEQ ID NO: 13 - DNA, 20 bp, synthetic construct
  source 1..20: mol_type = other DNA
agctcctcgcccttgctcac

SEQ ID NO: 14 - DNA, 26 bp, synthetic construct
  source 1..26: mol_type = other DNA
acttgaagctcctcgcccttgctcac

SEQ ID NO: 15 - DNA, 26 bp, synthetic construct
  source 1..26: mol_type = other DNA
gatcagagctcctcgcccttgctcac

SEQ ID NO: 16 - DNA, 26 bp, synthetic construct
  source 1..26: mol_type = other DNA
tagcttagctcctcgcccttgctcac

SEQ ID NO: 17 - DNA, 39 bp, synthetic construct
  source 1..39: mol_type = other DNA
tgtgaacattatcggcatcgctcgtggcgagcgtaatct

SEQ ID NO: 18 - DNA, 38 bp, synthetic construct
  source 1..38: mol_type = other DNA
cgatgccgataatgttcacatcagagcagtccttcacg

SEQ ID NO: 19 - DNA, 39 bp, synthetic construct
  source 1..39: mol_type = other DNA
taaggcaatcgtggtccttgcaaatttaaacgtggggtt

SEQ ID NO: 20 - DNA, 38 bp, synthetic construct
  source 1..38: mol_type = other DNA
caaggaccacgattgccttatactttacaatcatcttg